(19) United States
(12) Patent Application Publication
(10) Pub. No.: US 2025/0049962 A1
(43) Pub. Date: Feb. 13, 2025

(54) NEAR-INFRARED FLUORESCENT CONTRAST BIOIMAGING AGENTS FOR IMAGING OF SENTINEL LYMPH NODES

(71) Applicant: Curadel Surgical Innovations, Inc., Natick, MA (US)

(72) Inventor: John V. Frangioni, Weston, MA (US)

(21) Appl. No.: 18/719,519

(22) PCT Filed: Dec. 14, 2022
(86) PCT No.: PCT/US2022/052763

§ 371 Date: Jun. 13, 2024

Related U.S. Application Data
(60) Provisional application No. 63/290,286, filed on Dec. 16, 2021.

(51) Int. Cl. A61K 49/00; G01N 21/64

(52) U.S. Cl. CPC A61K 49/0032; G01N 21/6428; A61K 2123/00; G01N 2021/6439

(57) ABSTRACT

The instant invention provides near-infrared fluorescent biological contrast agents having higher hydrophilicity and solubility than standard imaging agents and methods of using them for the imaging and mapping of sentinel lymph nodes.

### CROSS-REFERENCE TO RELATED APPLICATIONS

This application claims priority to and the benefit of U.S. Provisional Application No. 63/290,286, filed Dec. 16, 2021, the entire contents of each are herein incorporated by reference in their entirety.

### BACKGROUND

Near infrared (NIR) fluorescence has potential importance in the medical field, particularly in in vitro diagnostics, in vivo diagnostics, and image-guided surgery. However, the availability of suitable fluorophores as imaging agents has been a primary hindrance. To be viable, ideal NIR fluorophores should have good optical properties as well as superior physicochemical properties with respect to solubility, biodistribution, targeting, and clearance. Most current fluorophores contemplated for use as imaging agents fail in connection with their physicochemical properties. For example, known fluorophores suffer from failure to adequately accumulate at the target to be imaged (i.e., low signal), resulting in a low signal-to-background ratio (SBR), or exhibit significant non-specific background uptake in normal tissues (i.e., high background), also resulting in a low SBR.

In particular, the mapping of sentinel lymph nodes poses additional challenges. Sentinel lymph node biopsy (SLB or SLN biopsy) allows selective, minimally invasive access for assessment of the regional lymph node status with malignant tumors. The first draining lymph note, the “sentinel”, represents an existing or non-existing tumor of an entire lymph node region. The method has been validated using radionuclides and/or blue dye for breast cancer, malignant melanoma, as well as and also gastrointestinal tumours, and provides a satisfactory detection rate and sensitivity. For the SLB, a reduced mortality has been observed in comparison with complete lymph node dissection, but the methods have disadvantages with regard to availability, application, disposal of the radionuclide, and the risk of anaphylaxis (up to 1%) for the blue dye.

Indocyanine green (ICG) is a fluorescent dye which is administered intravenously and, depending on liver performance, is eliminated from the body with a half life of about 3 to 4 minutes. ICG is metabolized in the liver and only excreted via the liver and bile ducts. Since it is not absorbed by the intestinal mucous membrane, the toxicity can be classified as low. Nevertheless, ICG is known to decompose into toxic waste materials under the influence of UV light, creating a number of unknown substances. Side effects such as anaphylactic shock, hypotension, tachycardia, dyspnea and urticaria occur in rare cases, though the risk of severe side-effects rises in patients with chronic kidney impairment.

ICG is currently used for SLN mapping and is thought to be effective because of its hydrophobic nature. Nevertheless, ICG is limited biological application due to its poor aqueous stability in vitro, concentration-dependent aggregation, rapid elimination from the body, and lack of target specificity. Other compounds have been shown to be useful for SLN mapping, such as those disclosed in WO2015066290A1. Nevertheless, the compounds described therein for SLN mapping are also hydrophobic in nature and are difficult to formulate for biological application.

As such, there is a current need for new and improved NIR fluorescent imaging agents for SLN mapping, particularly those that are readily soluble, equilibrate rapidly between the intravascular and extravascular spaces, target various cells, tissues, or organs with high sensitivity and specificity, and are eliminated efficiently from the body if not targeted. The imaging agents of the invention are directed toward these and other needs.

### SUMMARY

The present invention is directed, at least in part, to near-infrared fluorescent contrast agents and methods of using them.

In one aspect, the near-infrared fluorescent contrast agent is 4-[(2Z)-2-[(2E,4E)-5-[3,3-dimethyl-1-(4-sulfobutyl) indol-1-ium-2-yl]penta-2,4-dienylidene]-3,3-dimethylindol-1-yl]butane-1-sulfonic acid:

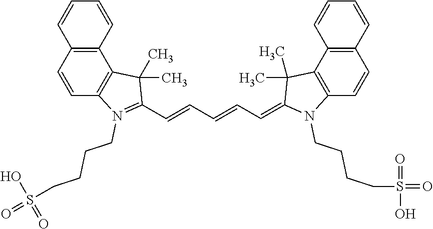

This compound is also referred to as MHI85, CUR-438, CID: 87460681, SCHEMBL2969196, or indocyanine blue (ICB).

In certain embodiments, the imaging agent has peak absorbance at about 600 nm to 900 nm.

In certain embodiments, the tissue or cells is imaged ex vivo, e.g. for in vitro diagnostic applications.

In another aspect, the invention provides a method of imaging sentinel lymph nodes (i.e. sentinel lymph node tissue, cells, vessels and/or lumens) comprising: (a) contacting one or more sentinel lymph nodes with ICB; (b) irradiating the cells at a wavelength absorbed by ICB; (c) and detecting a signal from ICB, thereby imaging the sentinel lymph node(s)

In certain embodiments, the compounds of the invention accumulate in a tissue or organ but do so extracellularly. For example, a compound injected sub-dermally may enter the lymphatic channels and flow to a sentinel lymph node where it may be trapped in the extracellular space rather than, or in addition to, entrapment within cells of the 1 sentinel lymph node.

In certain embodiments, the compounds of the invention may be modified to include a polyethylene glycol group. Such PEGylated compounds may be branched or linear. In certain embodiments, the linear PEGylated compounds are in the range of about 20 kDa to about 60 kDa.

In certain embodiments, ICB may be conjugated covalently or non-covalently to other molecules, either to improve targeting of the NIR fluorophore or to co-localize other functional molecules.

In some embodiments, ICB can be conjugated to a metal chelator agent for use in single-photon emission computed tomography (SPECT) or positron emission tomography (PET) or in magnetic resonance imaging (MRI). In certain embodiments, the metal chelator agent is a DOTA, DTPA, hydrazinonicotinic acid (HYNIC), or desferoxime, or a derivative thereof. In particular embodiments, the metal atom is selected from the group including, but not exclusively, Zr-89, Ga-68 and Rb-82, and the signal is detected by positron emission tomography; the metal atom is selected from the group including, but not exclusively, of Tc-99m, Lu-177, and In-111, and the signal is detected by single-photon emission computed tomography; or the metal atom is a lanthanide selected from the group including, but not exclusively, Gd, Eu, Y, Dy and Yb, and the signal is detected by magnetic resonance imaging.

In some embodiments, ICB can be conjugated to a therapeutic, such as a radioisotope, cytotoxin, or immune modulator, such that the targeting ability of the compound concentrates the therapeutic in the cell, tissue, organ, or lumen of interest.

### BRIEF DESCRIPTION OF THE DRAWINGS

Figures depicting the imaging of sentinel lymph nodes at 700 nm and 800 nm using indocyanine blue, indocyanine green, and MHI86 (Image without irradiation, NIR irradiated image, overlay of both)

FIG. 1—depicts the imaging of SLN identification at 700 nm using MHI86 (Image without irradiation, NIR irradiated image, overlay of both)

FIG. 1—depicts the imaging of SLN identification at 700 nm using MHI86 (Image without irradiation, NIR irradiated image, overlay of both)

FIG. 1—depicts the imaging of SLN identification at 700 nm using ICB and ICG (Image irradiated at 700 nm, NIR irradiated image, overlay of both)

FIG. 2—depicts the imaging of SLN identification at 700 nm using SLN700 and ICG (Image irradiated at 700 nm, NIR irradiated image, overlay of both)

FIG. 3—depicts the imaging of SLN identification at 700 nm using SLN700 and ICB (Image irradiated at 700 nm, NIR irradiated image, overlay of both)

FIG. 4—depicts the imaging of SLN identification after peri-tail intradermal injection using SLN700 (at 700 nm), ICB (referred to as CUR-438—at 700 nm), and ICG (at 800 nm)

FIGS. 5a, 5b, and 5c—depicts the biodistribution of SLN700, ICB (referred to as CUR-438), and ICG after 4 hours post intravenous injection.

FIG. 6—depicts the imaging of SLN identification at 700 nm using MHI86 (Image without irradiation, NIR irradiated image, overlay of both)

### ABBREVIATIONS USED

LN: lymph node
SLN: sentinel lymph node
SBR: signal-to-background ratio

### DETAILED DESCRIPTION

It has been found that compounds with absorption and/or emission in the near infrared (NIR) have desirable properties with respect to in vivo biodistribution and clearance, uptake and retention by cells, tissues, and/or organs of interest, and the imaging thereof. Such agents are compatible with Channel 1 (≈660 nm excitation; ≈700 nm emission) or Channel 2 (≈760 nm excitation; ≈800 nm emission) of the FLARE™ Imaging System, which permits color video and NIR fluorescence to be acquired simultaneously, thus providing real-time image-guidance to surgeons and others about target location.

### Definitions

The following definitions will be useful in understanding the instant invention.

Throughout the entire specification, including the claims, the following terms shall have the indicated meanings. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity

A/an: The articles “a” and “an” as used herein mean one or more when applied to any feature in embodiments and implementations of this disclosure described in the specification and claims. The use of “a” and “an” does not limit the meaning to a single feature unless such a limit is specifically stated. The term “a” or “an” entity refers to one or more of that entity. As such, the terms “a” (or “an”), “one or more” and “at least one” can be used interchangeably herein.

About: As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data. All numerical values within the detailed description and the claims herein are modified by “about” or “approximately” the indicated value, and take into account experimental error and variations that would be expected by a person having ordinary skill in the art.

And/or: The term “and/or” placed between a first entity and a second entity means one of (1) the first entity, (2) the second entity, and (3) the first entity and the second entity. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements). As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”.

Comprising: In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. Any device or method or system described herein can be comprised of, can consist of, or can consist essentially of any one or more of the described elements.

In particular, as used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

Ranges: Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and about 200, but also to include individual sizes such as 2, 3, 4, etc. and sub-ranges such as 10 to 50, 20 to 100, etc. Similarly, it should be understood that when numerical ranges are provided, such ranges are to be construed as providing literal support for claim limitations that only recite the lower value of the range as well as claims limitation that only recite the upper value of the range. For example, a disclosed numerical range of 10 to 100 provides literal support for a claim reciting “greater than 10” (with no upper bounds) and a claim reciting “less than 100” (with no lower bounds). In the figures, like numerals denote like, or similar, structures and/or features; and each of the illustrated structures and/or features may not be discussed in detail herein with reference to the figures. Similarly, each structure and/or feature may not be explicitly labeled in the figures; and any structure and/or feature that is discussed herein with reference to the figures may be utilized with any other structure and/or feature without departing from the scope of the present disclosure.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish, parasites, microbes, and the like.

As used herein, the term “administration” or “administering” of the subject compound refers to providing a compound of the invention and/or prodrugs thereof to a subject in need of diagnosis or treatment.

As used herein, the term “carrier” refers to chemical compounds or agents that facilitate the incorporation of a compound described herein into cells or tissues.

As used herein, the term “acceptable” with respect to a formulation, composition or ingredient, as used herein, means having no persistent detrimental effect on the general health of the subject being treated.

As used herein, the term “diluent” refers to chemical compounds that are used to dilute a compound described herein prior to delivery. Diluents can also be used to stabilize compounds described herein.

Other definitions appear in context throughout the disclosure.

### Compounds and Compositions

It has been found that certain compounds are useful as near-infrared absorbing and/or fluorescing biological contrast agents.

In one aspect, the near-infrared fluorescent contrast is In one aspect, the near-infrared fluorescent contrast agent is 4-[(2Z)-2-[(2E,4E)-5-[3,3-dimethyl-1-(4-sulfobutyl) indol-1-ium-2-yl]penta-2,4-dienylidene]-3,3-dimethylindol-1-yl]butane-1-sulfonic acid:

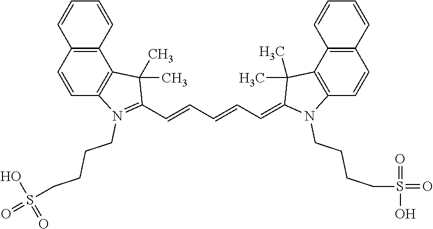

or a salt, solvate, hydrate, polymorph, prodrug, or stereoisomer thereof.

This compound is also referred to as MHI85, CUR-438, CID: 87460681, SCHEMBL2969196, or indocyanine blue (ICB).

In certain embodiments, ICB can absorb light at different wavelengths in the near-infrared region. Specifically, in some embodiments, the compounds of the invention absorb light in the 660-720 nm range. In other embodiments, the compounds of the invention absorb light in the 760-820 nm range.

Due to it increased hydrophilicity, as compared to ICG, ICB is highly soluble in water and other carriers. The increased hydrophilicity of ICB is due to the shorter alkynylene chain connecting the benzoindole groups. In addition, the inclusion of sulfobutyl groups also greatly adds to the hydrophilicity of ICB. It has been unexpectedly and surprisingly found that, despite its relative lack of hydrophobicity. ICB is readily retained by sentinel lymph nodes in amounts effective for SLN mapping.

In certain embodiments, ICB comprises a radioisotope having a single-photon or positron emission decay mode and suitable for detection by single-photon emission tomography (SPECT) or positron emission tomography (PET) in addition to its detection via optical properties (i.e., absorption and/or fluorescence). Examples of suitable radioisotopes include C-11 and F-18. Such isotopes can be incorporated into a compound of the invention, e.g., by use of appropriate isotopically-enriched reagents during synthesis of the compound. Additional useful radiotracers, such as Ga-68 Zr-89, or Rb-82 (PET), or Tc-99m (SPECT), can be attached to the compound through a radiometal chelator such as 1,4,7,10-tetraazacyclododecane-1,4,7,10-tetraacetic acid (DOTA), diethylene triamine pentaacetic acid (DTPA), hydrazinonicotinic acid (HYNIC), or desferoxime, respectively (or derivatives thereof). Chelator moieties can be covalently attached to an oxazine compound, e.g., through a linking atom or group, e.g., by acylation of a hydroxyl group of a compound of Formula I-V with a carboxylate group of a chelator such as DOTA. By incorporation of an appropriate PET- or SPECT-detectable isotope, a compound according to the invention can be detected using SPECT or PET imaging (e.g., even when administered at a low dose), e.g., using a conventional SPECT or PET imaging system, while also being detectable optically (e.g., by fluorescence imaging), e.g., when administered at a higher dose. Dual-mode optical and SPECT or PET imaging is also possible using such compounds. Similarly, imaging by magnetic resonance imaging (MRI), including dual-mode optical/MRI imaging, can be performed by using a compound of the invention comprising a lanthanide (such as Yb³⁺, Dy³⁺ or Gd³⁺), e.g., by chelating the lanthanide ion using a suitable chelating moiety.

ICB can be prepared using a variety of methods, some of which are known in the art. For example, the compounds can be prepared using conventional methods of synthetic organic chemistry (see, e.g., Michael B. Smith, “March's Advanced Organic Chemistry: Reactions, Mechanisms, and Structure, 7th Edition”, Wiley (2013)).

For example, ICB can be synthesized using the syntheses described in scheme 1 shown below. General references for the syntheses described in the scheme 1 is found in Henary, M. et al. Bioorg. & Med. Chem. Let. 22, 242-1246, (2012), Henary, M. et al. J. Heterocycl. Chem. 46:84-87, (2009), Henary, M. et al. Dyes and Pigments. 99, 1107-1116 (2013), Henary, M. et al. Heterocycl. Commun. 19 (1), 1-11 (2013), Mojzych, M. et al. Topics in Heterocyclic, Springer-Verlag Berlin Heidelberg. 14, 1-9 (2008), Strekowski, L. et al. J. Org. Chem. 57, 4578-80 (1992), Halder, S. et. al. Eur. J. Med. Chem. 54, 647-59 (2012), Sakiko, A. et al. Chem.-A Eur. J., 15, 9191-9200 (2009), Chang, Y-T. et al. Chem. Commun. 47, 3514-3516 (2011), Myochin, T. J. Am. Chem. Soc. 134,13730-13737 (2012), Briza, T. et al. Chem. Comm. 16, 1901-1903 (2008), Chang, Y-T. et al. Chem. Commum. 46, 7406-7408 (2010), Zaheer, A., et al. Molecular Imag. 1 (4), 1536-0121 (2002), Misra, P. et al. J Nucl Med. 2007 August; 48 (8): 1379-89, and Humblet, V. et al, J Med Chem. 2009 Jan. 22; 52(2): 544-50.

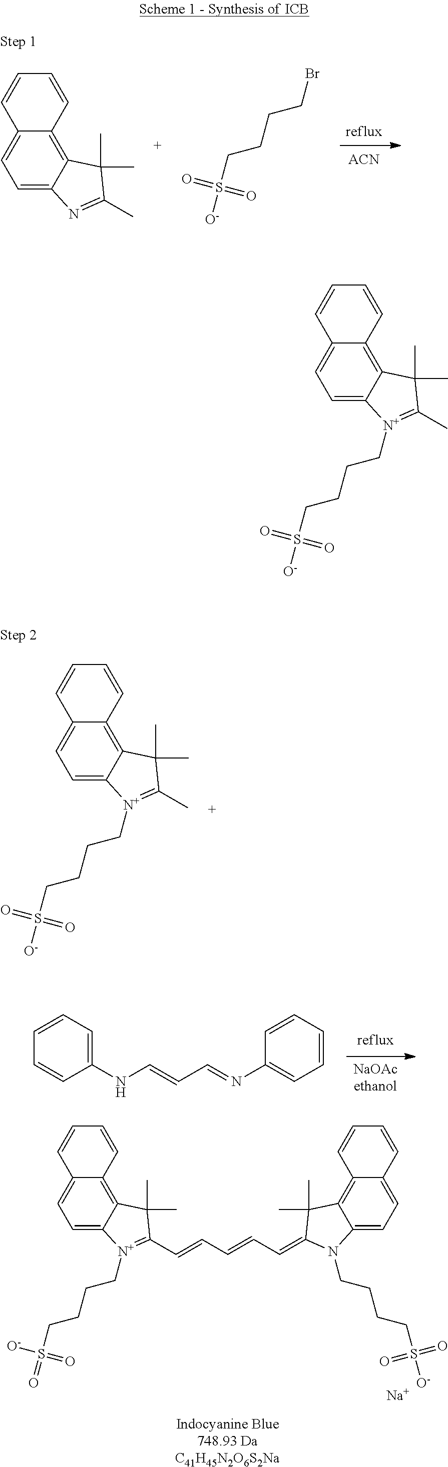

ICB can also be synthesized using the three-step synthesis described in Scheme 2 below:

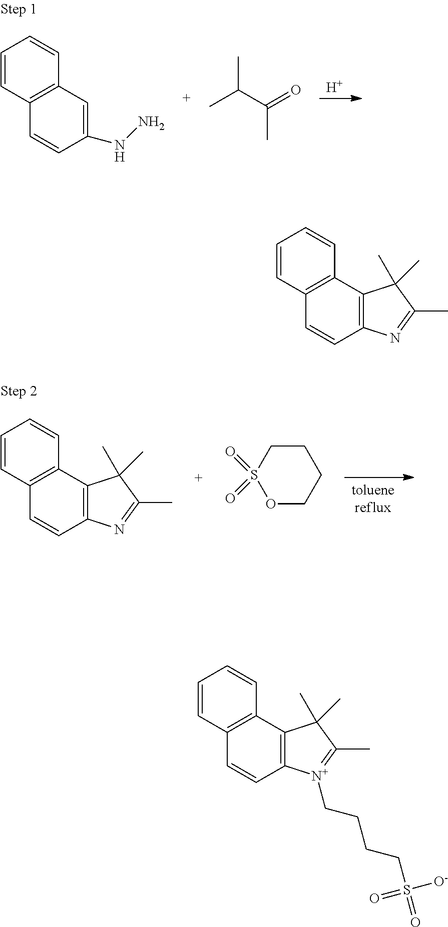

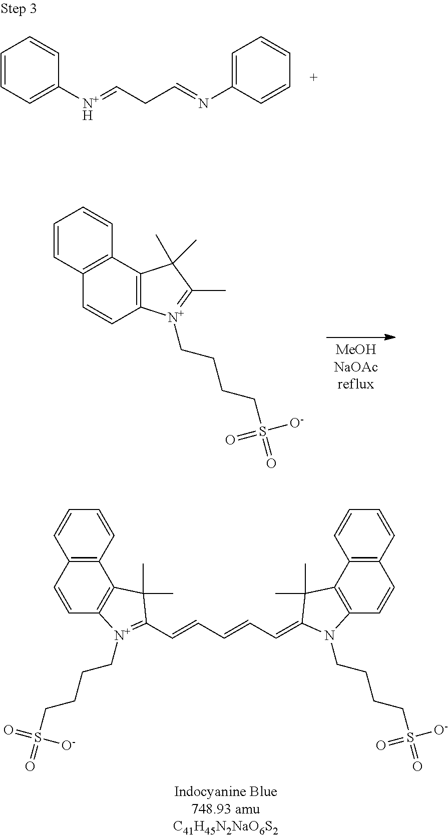

Further still, ICB can be also be synthesized using the methods disclosed in WO2015066290A1, EP2152810A1, US20080254546A1, US20100216182A1, U.S. Pat. Nos. 7,776,999B2, 8,354,499B2, WO2008127768A1, each of which are incorporated by reference.

ICB, including salts, solvates, hydrates thereof, can be synthesized using the methods outlined above or with modification of starting materials and other reagents as will be readily understood by one of ordinary skill in the art.

### Compositions

In another aspect, the invention provides pharmaceutical compositions of ICB, or pharmaceutically acceptable salts, solvates, or hydrates thereof.

For the therapeutic uses provided herein, ICB including pharmaceutically acceptable salts, solvates, N-oxides, prodrugs, or isomers thereof, is administered in therapeutically effective amounts either alone or as part of a pharmaceutical composition. Accordingly, provided herein are pharmaceutical compositions, which comprise at least ICB, pharmaceutically acceptable salts and/or solvates thereof, and one or more pharmaceutically acceptable carriers, diluents, adjuvant or excipients. The methods of administration of ICB or compositions of ICB include, but are not limited to, intravenous administration, inhalation, oral administration, rectal administration, parenteral, intravitreal administration, intratumoral administration, subcutaneous administration, intramuscular administration, intranasal administration, dermal administration, topical administration, ophthalmic administration, buccal administration, tracheal administration, bronchial administration, sublingual administration or optic administration. Compounds provided herein are administered by way of known pharmaceutical formulations, including tablets, capsules or elixirs for oral administration, suppositories for rectal administration, sterile solutions or suspensions for parenteral or intramuscular administration, lotions, gels, ointments or creams for topical administration, and the like.

In certain embodiments, ICB can be administered directly without any additional formulation. Nevertheless, because of its increased solubility, ICB can be readily dissolved in water, saline, 5% dextrose in water (D5W), or other carriers for administration.

The amount administered will vary depending on, among others, the tissue or organ to be imaged, the age and relative health of the subject, the potency of the compound administered, the mode of administration and the like. In some embodiments ICB is administered as an injection of 0.1 mL-5 ml of a 1.0-5.0 mM solution. In certain embodiments, ICB is administered as an injection of 1.0 ml-2.5 ml of a 1.0-3.0 mM solution. In specific embodiments, ICB is administered as an injection of 1.0 mL of a 2.5 mM solution, equivalent to 2.5 μmoles or 1.57 mg of compound.

Pharmaceutically acceptable salt forms include pharmaceutically acceptable acidic/anionic or basic/cationic salts. Pharmaceutically acceptable acidic/anionic salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphosphate, polygalacturonate, salicylate, stearate, subacetate, succinate, sulfate, hydrogensulfate, tannate, tartrate, teoclate, tosylate, and triethiodide salts. Pharmaceutically acceptable basic/cationic salts include, the sodium, potassium, calcium, magnesium, diethanolamine, N-methyl-D-glucamine, L-lysine, L-arginine, ammonium, ethanolamine, piperazine and triethanolamine salts.

A pharmaceutically acceptable acid salt is formed by reaction of the free base form of ICB with a suitable inorganic or organic acid including, but not limited to, hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid. A pharmaceutically acceptable acid addition salt of a compound of Formula I can comprise or be, for example, a hydrobromide, hydrochloride, sulfate, nitrate, phosphate, succinate, maleate, formarate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g., 2-naphthalenesulfonate) or hexanoate salt.

The free acid or free base forms of ICB may be prepared from the corresponding base addition salt or acid addition salt form, respectively. For example ICB in an acid addition salt form may be converted to the corresponding free base form by treating with a suitable base (e.g., ammonium hydroxide solution, sodium hydroxide, and the like). ICB in a base addition salt form may be converted to the corresponding free acid by treating with a suitable acid (e.g., hydrochloric acid, etc.).

Prodrug derivatives of ICB may be prepared by methods known to those of ordinary skill in the art (e.g., for further details see Saulnier et al., Bioorg. Med. Chem. Letters, 1994, 4, 1985; the entire teachings of which are incorporated herein by reference).

Protected derivatives of ICB may be prepared by means known to those of ordinary skill in the art. A detailed description of techniques applicable to the creation of protecting groups and their removal can be found in T. W. Greene, “Protecting Groups in Organic Chemistry,” 3rd edition, John Wiley and Sons, Inc., 1999, the entire teachings of which are incorporated herein by reference.

Suitable pharmaceutically acceptable carriers, diluents, adjuvants, or excipients for use in the pharmaceutical compositions of the invention include tablets (coated tablets) made of for example collidone or shellac, gum Arabic, talc, titanium dioxide or sugar, capsules (gelatin), solutions (aqueous or aqueous-ethanolic solution), syrups containing the active substances, emulsions or inhalable powders (of various saccharides such as lactose or glucose, salts and mixture of these excipients with one another) and aerosols (propellant-containing or -free inhale solutions).

Excipients which may be used include, for example, water, pharmaceutically acceptable organic solvents such as paraffins (e.g., petroleum fractions), vegetable oils (e.g. groundnut or sesame oil), mono- or polyfunctional alcohols (e.g., ethanol or glycerol), carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone) and lubricants (e.g., magnesium stearate, talc, stearic acid and sodium lauryl sulphate).

Exemplary methods for preparing the compounds of the invention are described herein, including in the Examples.

### Methods

The present invention features various methods using the near-infrared fluorescent biological contrast agent described herein.

In one aspect, the invention provides a method of imaging biological tissue or cells, the method comprising:
(a) contacting the biological tissue or cells with indocyanine blue (ICB) or a pharmaceutically acceptable salt, solvate, or hydrate thereof;
(b) irradiating the biological tissue or cells at a wavelength absorbed by ICB;
(c) and detecting a signal from the ICB, thereby imaging the biological cells.

The signal may be in the form of absorption, such as occurs during photoacoustic imaging. Alternatively, the imaging agent can have a SBR suitable to permit fluorescence detection. SBR is a measure of the intensity of the fluorescent signal obtained from a target (peak signal) over the measure of the intensity of the fluorescent signal obtained nearby the target (background signal), the target being the tissues, cells, space targeted by the imaging agent. SBR measurements can be readily obtained through routine measurement procedures. For fluorescent imaging systems, and other optical-type systems, digital images recording optical signals of the target facilitate SBR measurement. Higher SBR values are more desirable, resulting in greater resolution of the imaged tissues. In some embodiments, the imaging agents achieve an SBR of at least about 1.1 (i.e., peak signal is at least 10% over background). In further embodiments, the imaging agents achieve an SBR of at least about 1.2, at least about 1.3, at least about 1.4, at least about 1.5, at least about 1.6, at least about 1.7, at least about 1.8, at least about 1.9, or at least about 2.0. In yet further embodiments, the imaging agents achieve an SBR of about 1.1 to about 50, about 1.5 to about 30, about 2.0 to about 20, about 2.0 to about 5.0, or about 5.0 to about 10.

In certain embodiments, the imaging agent is administered directly to a subject or biological system for the imaging of the targeted cells.

In other embodiments, reactive derivates of the imaging agent are used to label chemical and biological molecules for further study. Certain molecules which may be labeled using reactive derivatives of the imaging agents of the invention include small molecules (including pharmaceutical, neutraceutical, therapeutic and diagnostic compounds, proteins, peptides, peptidomimetics, antibodies, vaccines, and other chemical and biological molecules which may be of interest in studying by NIR imaging. In such embodiments, the imaging agent of the invention is reacted with the chemical or biological molecule to produce a labeled agent molecule which may then be administered to a subject or biological system for imaging as described herein.

The steps of irradiating the tissue or cells at a wavelength absorbed by the imaging agent, and detecting an optical signal from the irradiated tissue or cells, thereby imaging the tissue or cells, can be performed using an imaging system such as the FLARE™ Image-Guided Surgery System, which is a continuous-wave (CW) intraoperative imaging system that is capable of simultaneous, real-time acquisition and display of color video (i.e., surgical anatomy) and two channels of invisible NIR fluorescent (700 nm and 800 nm) light (see, e.g., Gioux et al., Mol. Imaging. 9 (5): 237-255 (2010) and U.S. Pat. No. 8,473,035 to Frangioni, for a description of suitable systems). With FLARE™ and other NIR fluorescence imaging systems, contrast agent emission wavelength in the 800-850 nm range (Channel 2 of FLARE™) is preferred whenever possible because of lower autofluorescence and lower attenuation from both absorbance and scatter when compared to emission near 700 nm. Nevertheless, fluorophores emitting within Channel 1 (≈700 nm) of the FLARE™ imaging system still retain the benefits of NIR fluorescence imaging, including detection of nerves and other targets below the surface of blood and tissue.

In some embodiments, the imaging agent can be formulated into pharmaceutically acceptable formulations and administered intravenously to an organism for imaging. The dosed organism can be imaged using, for example, the FLARE™ system. The imaging system can irradiate the dosed organism with radiation absorbed by the imaging agent, and detect optical signals, such as NIR fluorescence, emanating from the targeted portions of the organism containing the imaging agent. The detected signals can be recorded and analyzed by obtaining digital images or video of the subject organism, thereby facilitating diagnostic procedures and image-guided medical techniques.

The invention also provides methods of performing image-guided surgery, the methods comprising imaging cells, tissues, or organs according to a method described herein, and then performing surgery such that the targets are either removed or are preserved, depending on the goals of the surgical intervention. In certain preferred embodiments, the contrast agent is injected intravenously to ensure that all targets are labeled, and imaging is performed after sufficient time has passed for biodistribution to nerves and clearance of surrounding background signal.

In certain embodiments, the targets are biological tissues or organs. In specific embodiments, the targets are lumens, such as sentinel lymph nodes.

It should also be noted that although the examples given below are for in vivo imaging, which represents the most difficult situation because properties such as biodistribution and clearance are dictated in large part by the organism, those skilled in the art will recognize that these same contrast agents can be used for any type of in vitro assay, such as immunohistology, detection of targets in blood or bodily fluid samples, etc. using the same principles of contact with the medium, washout of unbound dose, and detection of a signal derived from absorption, fluorescence emission and/or radioactive emission.

### Sentinel Lymph Nodes:

Sentinel lymph node mapping has revolutionized the treatment of breast cancer and melanoma. However, 20-25% of patients are found to have tumor cells in their sentinel lymph node and therefore require a completion lymphadenectomy, i.e., removal of all the lymph nodes in the basin. Finding all lymph nodes in an area of the body is extremely difficult to do.

Sentinel lymph node agents are injected in and around a tumor and quickly flow to the first lymph node that drains the tumor, called the sentinel lymph node (SLN).

As such, in one aspect, the invention provides a method for imaging sentinel lymph nodes, the method comprising:
(a) contacting the sentinel lymph node with indocyanine blue (ICB) or a pharmaceutically acceptable salt, solvate, or hydrate thereof;
(b) irradiating the tissue at a wavelength absorbed by ICB;
(c) and detecting a signal from ICB, thereby imaging the sentinel lymph node.

In a particular embodiment, for use in sentinel lymph node imaging the irradiating wavelength is in the 660-700 nm range.

In another particular embodiment, for use in sentinel lymph node imaging the irradiating wavelength is in the 760-800 nm range.

In certain embodiments, ICB can be used in mapping sentinel lymph nodes. In still other embodiments, the compounds of the invention can be used in identifying breast cancer. In such embodiments, the sentinel lymph nodes of the organism are imaged to provide a map of the sentinel lymph nodes. A sample of the sentinel lymph nodes are then removed by biopsy and the nodes removed are examined to determine if breast cancer cells are present. In such embodiments, the methods in which the removed nodes are examined is not particularly limited and would be readily understood by one of ordinary skill in the art of diagnosing breast cancer.

### Angiography:

Mapping the vasculature in and around lymph nodes during surgery can often assist with resection. The mapping of vasculature, called angiography, is used to visualize the inside, or lumen, of blood vessels and organs of the body, with particular interest in the arteries, veins, and the heart chambers.

A NIR fluorophore injected into the bloodstream can act as an angiographic agent because during the first 8 seconds after intravenous injection there is a rapid arterial flush (≈1 second), a rapid capillary flush (2-3 seconds), a rapid venous flush (≈1-2 seconds), then minutes of clearance from the tissue. The first 8 seconds thus provides a “map” of the circulation in the tissue. NIR angiography is important for imaging the perfusion of skin during plastic and reconstructive surgery and the anastomoses of bowel during gastrointestinal surgery. In general, NIR angiography agents are those that are cleared rapidly from the blood into either urine or bile.

As such, in one aspect, the invention provides a method of performing angiography in an organism, the method comprising:
(a) administering an imaging composition to an organism, wherein the imaging composition comprises an imaging agent, and wherein the administering comprises contacting blood vessels, lymph nodes, and/or coronary lumens of the organism with the imaging agent,
wherein the imaging agent is

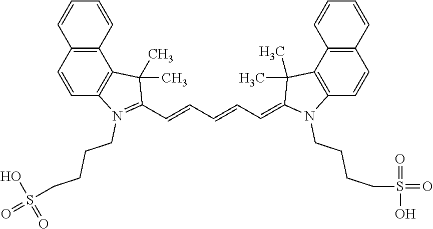

or a salt, solvate, hydrate, polymorph, or prodrug, thereof;
(b) irradiating the blood vessels, lymph nodes, and/or coronary lumens at a wavelength absorbed by the imaging agent; and
(c) detecting a signal from the imaging agent to identify the blood vessels, lymph nodes, and/or coronary lumens.

In another aspect, the invention provides a method for imaging tissue perfusion, the method comprising:
(a) contacting the blood with an imaging composition, wherein the imaging composition comprises an imaging agent, and wherein the imaging agent is

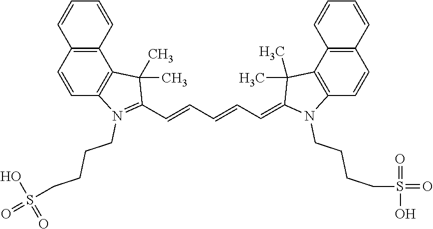

or a salt, solvate, hydrate, polymorph, or prodrug, thereof;
(b) irradiating the blood vessels and surrounding tissue at a wavelength absorbed by the imaging agent;
(c) and detecting a signal from the compound, thereby imaging the distribution and clearance of the imaging agent in the tissue over time.

### EXAMPLES

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1—Synthesis Examples—Compound and Composition

Indocyanine Blue is prepared according to the methods described in Scheme 1.

Compositions of Indocyanine Blue are prepared by dissolving Indocyanine Blue in water or in D5W at a concentration of 2.5 mM.

### Example 2—Optical Properties/SLN Mapping

### Optical Property Measurements

Absorbance and fluorescence emission spectra were measured using fiber optic HR2000 absorbance (200-1100 nm) and USB2000FL fluorescence (350-1000 nm) spectrometers (Ocean Optics, Dunedin, FL). Excitation was provided by a 532 nm green laser pointer (Opcom Inc., Xiamen, China) set to 5 mW, a 655 nm red laser pointer (Opcom Inc., Xiamen, China) set to 5 mW, or a 770 nm NIR laser diode light source (Electro Optical Components, Santa Rosa, CA) set to 10 mW and coupled through a 300 μm core diameter, NA 0.22 fiber (Fiberguide Industries, Stirling, NJ). In silico calculations of the partition coefficient (logD at pH 7.4) and surface molecular charge and hydrophobicity were calculated using MarvinSketch 5.2.1 by taking major microspecies at pH 7.4 (ChemAxon, Budapest, Hungary).

### NIR Fluorescence Imaging System

Neae Infrared fluorescence imaging can be achieved using a number of commercially available imaging systes. For example, the dual-NIR channel FLARE imaging system has been described in detail. It provides simultaneous illumination with white light (400-650 nm) at 40,000 lx, 660 nm NIR Channel 1 excitation at 4 mW/cm² and 760 bmm NIR Channel 2 excitation at 10 mW/cm². Color and two independent NIR fluorescence emission images (≈700 nm for Channel 1 and ≈800 nm for Channel 2) were acquired simultaneously with custom software at rates up to 15 Hz over a 15 cm diameter field of view. NIR fluorescence from Channel 1 was pseudo-colored in red and NIR fluorescence from Channel 2 was pseudo-colored in lime green prior to merger with the color video image. The imaging system was positioned at a distance of 18 inches from the surgical field.

### Animal Models and Intraoperative NIR Fluorescence Imaging

Animals were housed in an AAALAC-certified facility. Animal studies were performed under the supervision of Beth Israel Deaconess Medical Center's Institutional Animal Care and Use Committee (IACUC) in accordance with approved institutional protocols (#101-2011 for rodents and #046-2010 for pigs).

Initial in vivo screening occurred in mice, rats, and pigs In the animal studies described below, either sex of 25 g CD-1 mice (Charles River Laboratories, Wilmington, MA) and either sex of 250 g Sprague-Dawley (SD) rats (Taconic Farms, Germantown, NY) were used after anesthetizing with 100 mg/kg ketamine and 10 mg/kg xylazine intraperitoneally (Webster Veterinary, Fort Devens, MA). Either sex of Yorkshire pigs (E. M. Parsons and Sons, Hadley, MA) averaging 35 kg were induced with 4.4 mg/kg intramuscular Telazol™ (Fort Dodge Labs, Fort Dodge, IA), intubated, and maintained with 2% isoflurane (Baxter Healthcare Corp., Deerfield, IL). Following anesthesia, a 16G central venous catheter was inserted into the external jugular vein, and saline was administered as needed. Electrocardiogram, heart rate, pulse oximetry, and body temperature were monitored throughout surgery.

To screen the optimum targeted contrast agent, 2-200 nmol of each NIR fluorophore was injected intravenously in CD-1 mice and sacrificed animals 1-4 h post-injection (n>3). Target tissues/organs were observed at indicated time points such as 0, 5, 10, 15, 30, 60, 120, 180, and 240 min with the FLARE™ imaging system. After intraoperative imaging, animals were sacrificed, and the target tissue and other major organs including heart, lung, liver, spleen, pancreas, kidneys, duodenum, intestine, and muscle were resected to quantify biodistribution and clearance. For rats, an optimized dose (10-1000 nmol) was injected depending on the targeting purpose, and targeting and biodistribution were observed 4 h post-injection (n>3). For the large animal study, the appropriate dose was calculated based on the previous dose dependence study in the small animal study. To confirm the drug kinetics in large animals, 0.5-10 μmol of the NIR fluorescence was injected through the external jugular vein (n>3). Then the target tissue and surrounding organ were imaged at the indicated time points (0, 1, 5, 10, 15, 30, 60, 90, 120, 180, and 240 min).

### Example 3—Comparative Example

To demonstrate the superior effects of ICB as a contrast agent, ICB was compared with previously known contrast agents indocyanine green (ICG) and SLN-700.

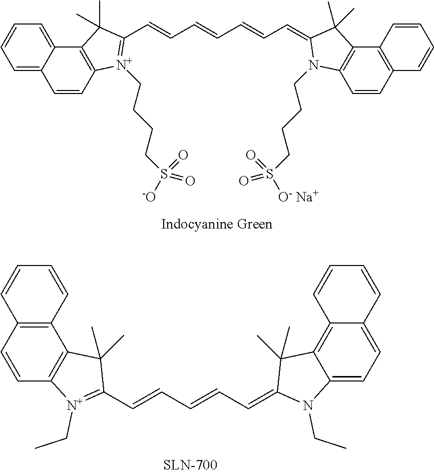

The experimental conditions were:
ICG: Reconstituted in water at 500 μM
SLN700: Reconstituted in 50% ethanol/50% water at 500 μM
CUR-438: Reconstituted in D5W at 500 μM

All foot pad injections and intravenous injections were 10 μL of each of these 500 μM concentrations for a total of 5 nmol per injection.

5 nmol of ICB, indocyanine green, and SLN-700 were injected via the footpad of CD-1 mice which were sacrificed immediately post-injection (n>3). Sentinel lymph nodes were observed a near infrared (NIR) imaging system.

The target tissue was exposed and the SLN was imaged for NIR fluorescence using dual channels [Channel 1 (700 nm) and Channel 2 (800 nm)] of a NIR imaging system.

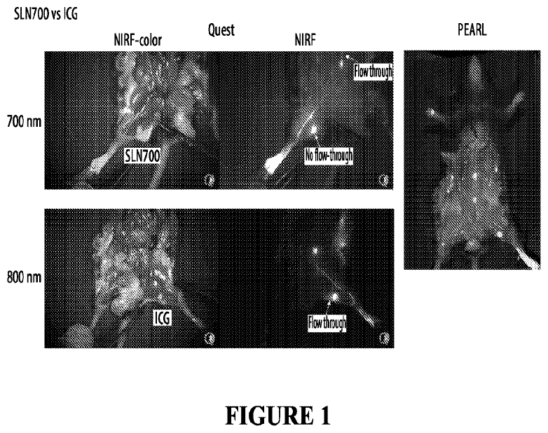

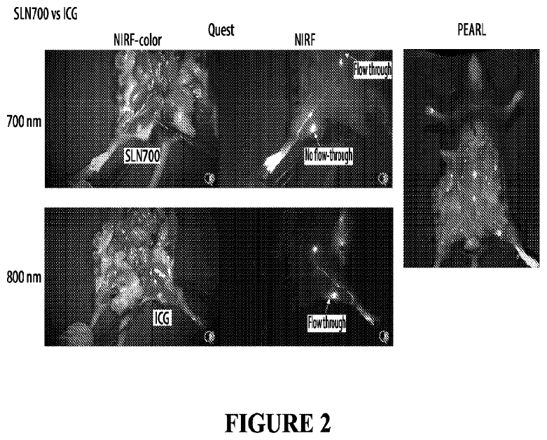

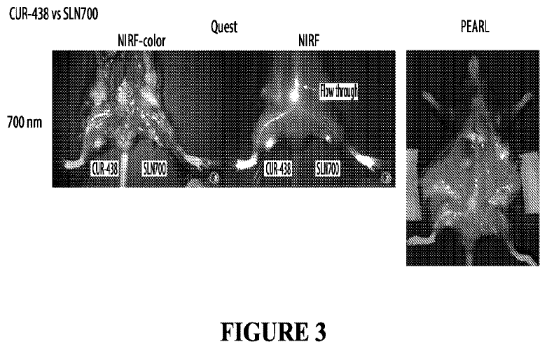

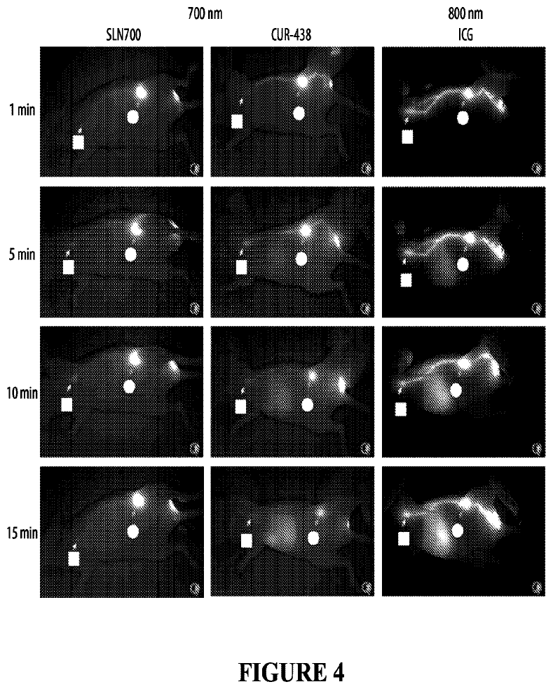

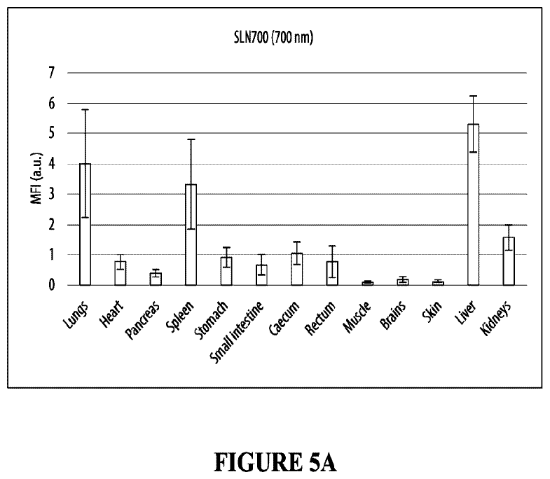

FIG. 1 shows a comparison of ICB with indocyanine green. FIG. 2 shows a comparison of SLN700 with indocyanine green. FIG. 3 shows a comparison of SLN 700 with ICB.

In particular, it was observed that at 15 minutes a post-injection, first-tier/second-tier signal-to-background ratio of 3.0±0.65 was observed for indocyanine green, 8.1±4.0 was observed for SLN700, 5.4±1.6 were observed for SLN700 and ICB, respectively. In addition, ICB demonstrates considerably less leakage over ICG or SLN700 as observed by the flow through of these components. In the ICB figures, the sentinel lymph nodes are clearly identified with little to no significant flow through. In addition, as compared to SLN700, the visualization of ICB is brighter allowing for easier identification during testing.

Visible liver (hepatic) clearance of ICB is also observed by the imaging. This was significantly more pronounced than in SLN700.

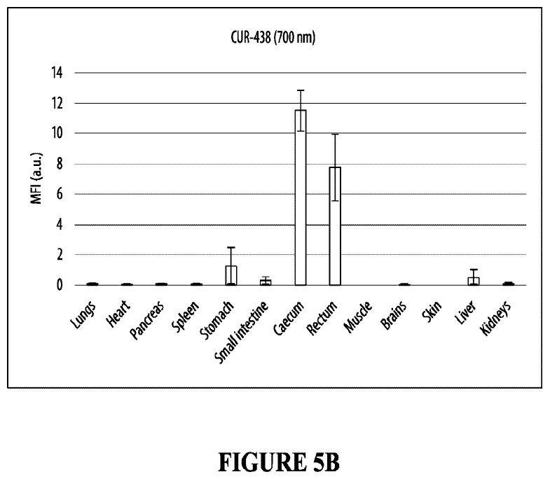

The same procedure was used using intradermal peri-tail injections to visual lymph flow until 15 minutes post injection at which point the animal was sacrificed. This procedure was performed with visualization at 700 nm for ICB and SLN700 and at 800 nm for ICG. This data is presented in FIG. 4.

The arrow with a square shows the second-tier (axial) lymph node. The arrow with the circle indicates the first-tier node. As can be seen from FIG. 4, ICG demonstrates significant LN skipping as compared to ICB or SLN700.

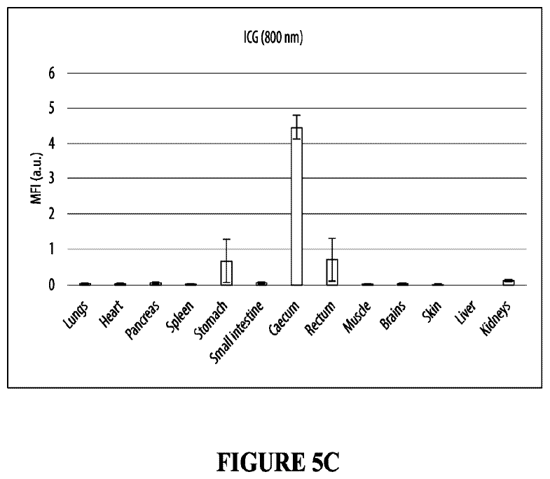

In order to show the biodistribution benefits of ICB, the same procedure was used using intravenous injections. After 4 hours animals were sacrificed, and the target tissue and other major organs including heart, lung, liver, spleen, pancreas, kidneys, duodenum, intestine, and muscle were resected to quantify biodistribution and clearance. A biodistribution analysis of all three dyes at 4 hours post iv injection. This data is presented in FIGS. 5A, 5B, and 5C.

Both ICG and ICB show enhanced signal in the large intestine, but to lesser extent in the stomach which suggests excretion of both dyes through the bile. Minimal signal was observed in the kidneys for ICB. In contrast, for SLN700, no direct evidence of bile excretion into the large intestine was observed, but both liver and kidney signals were enhanced. Spleen and lung signals were also high for SLN700. This is believed to be related to the high perfusion of both organs. From this data, it is clear that ICB can be completely cleared by the liver allowing for easier administration and tolerance while also increasing efficacy of mapping sentinel lymph nodes as compared to ICG or SLN700.

### Example 4—Large Animal Study

For the large animal study, the appropriate dose was calculated based on a previous dose dependence study in the a animal study. To confirm the drug kinetics in large animals, 0.5-10 μmol of the NIR fluorescence was injected through the external jugular vein (n>3). Then the target tissue and surrounding organ were imaged at the indicated time points (0, 1, 5, 10, 15, 30, 60, 90, 120, 180, and 240 min).

SLN: A 35 kg female pig having a chemically induced tumor is injected intratumorally at time zero with 5 nmol of indocyanine green dissolved in saline or D5W. After a waiting period of 5 min, the target tissue is exposed and the SLN is imaged for NIR fluorescence using Channel 1 (700 nm) or Channel 2 (800 nm) of the FLARE imaging system, respectively.

SLN: A 35 kg female pig having a chemically induced tumor is injected intratumorally at time zero with 5 nmol of indocyanine blue dissolved in saline or D5W. After a waiting period of 5 min, the target tissue is exposed and the SLN is imaged for NIR fluorescence using Channel 1 (700 nm) or Channel 2 (800 nm) of the FLARE imaging system, respectively.

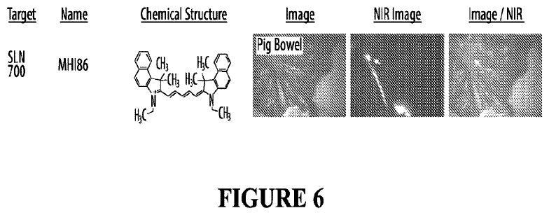

SLN: A 35 kg female pig was injected subcutaneously into bowel at time zero with 5 nmol of compound SML700 (700 nm) dissolved in saline or DSW. After a waiting period of 5 min, the target tissue was exposed and the SLN was imaged for NIR fluorescence using Channel 1 (700 nm) of the FLARE imaging system, respectively. As shown in FIG. 6, the SLN is highlighted with high contrast using this compound.

### Example 5—Hydrophobicity Measurement

Hydrophobicity of ICB, ICG, and MHI86 is determined by measuring the octanol-water partition coefficients (log P) of the agents. Negative log P values are characteristic of high water solubility. Such measurements may be made by methods described in Cumminh, H. and Rucker, C., “Octanol-Water Partition Coefficient Measurement by a Simple 1H NMR Method,” ACS Omega 2017 2 (9), 6244-6249.

### Example 6—Solubility Measurement

Indocyanine green has a rather poor aqueous solubility of 2.5 mg/mL. To achieve this solubility, iodide is added to a concentration of 1-5%,

SLN700 exhibits poor solubility in pure aqueous environments such as deionized water and PBS. For R&D purposes, the highest observable solubility is 66 mg/mL in DMSO. However, the contrast agent is soluble in mixtures of water and ethanol. The solubility of SLN700 in water:ethanol blends was complete by mixing excess contrast agent with premixed solvents. After mixing for an extended period, the solutions were filtered using a 0.45 um spin filter. The solvent of known volumes was then removed by evaporation and solubility was determined by measuring the remaining solids in the pre-measured evaporating flask. The results are shown below.

TABLE 1
Solubility of SLN700 in water:ethanol blends.

| Ethanol-Water | SLN700 Solubility | SLN700 Solubility |
|---|---|---|
| (% v/v) | (mg/mL) | (mM) |
| 0 | 0.0 | 0.00 |
| 25 | 0.0 | 0.00 |
| 50 | 0.9 | 1.41 |
| 75 | 1.6 | 2.51 |
| 100 | 2.5 | 3.91 |

In contrast to the above, indocyanine blue was tested both water and D5W and was found to have a solubility of at least 10 mg/mL in each case.

As can be seen from the data discussed above, ICB provides significantly superior results in solubility. This allows for significantly easier and safer formulation and administration of the dye to a subject as compared to ICG or SLN700.

### INCORPORATION BY REFERENCE

The entire contents of all patents, published patent applications and other references cited herein are hereby expressly incorporated herein in their entireties by reference.

### EQUIVALENTS

While the present disclosure has been described with respect to a number of embodiments and examples, those skilled in the art, having benefit of this disclosure, will appreciate that other embodiments can be devised which do not depart from the scope and spirit of the present disclosure.

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired products, additional ingredients may be added, and/or similar ingredients may be substituted for one or more of the ingredients described.

Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein.

What is claimed is:

1. A method of imaging sentinel lymph nodes, the method comprising:
(a) administering an imaging composition to an organism comprising the lymph nodes, wherein the imaging composition comprises an imaging agent, and wherein administering the imaging composition comprises contacting the sentinel lymph nodes with the imaging agent,
wherein the imaging agent is 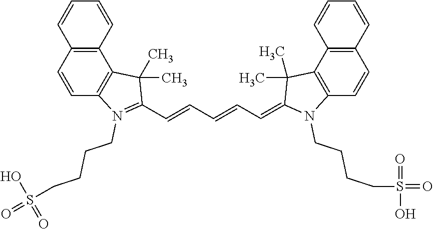
or a salt, solvate, hydrate, polymorph, or prodrug, thereof;
(b) irradiating the tissue, lumen, or cells of the sentinel lymph nodes at a wavelength absorbed by the imaging agent;
(c) and detecting a signal from the imaging agent, thereby imaging the lymph nodes.

2. The method of claim 1, wherein the organism is human.

3. The method of claim 1, wherein the imaging agent has peak absorbance at about 600 nm to 850 nm.

4. The method of claim 1, wherein the lymph nodes are imaged in vivo.

5. The method of claim 1, wherein the administering step is performed by intratumoral administration, subdermal administration, subcutaneous administration, parenchymal administration, or a combination thereof.

6. A method of mapping sentinel lymph nodes in an organism, the method comprising:
(a) administering an imaging composition to an organism comprising the sentinel lymph nodes, wherein the imaging composition comprises an imaging agent, and wherein the administering comprises contacting the sentinel lymph nodes with the imaging agent,
wherein the imaging agent is 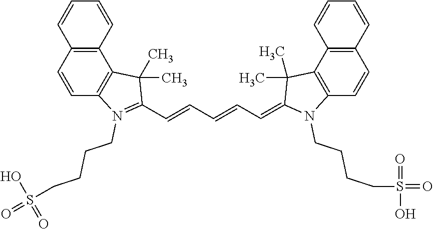
or a salt, solvate, hydrate, polymorph, or prodrug, thereof;
(b) irradiating the sentinel lymph nodes at a wavelength absorbed by the imaging agent;
(c) and detecting a signal from the imaging agent, thereby mapping the sentinel lymph nodes.

7. The method of claim 6, wherein the organism is human.

8. The method of claim 6, wherein the imaging agent has peak absorbance at about 600 nm to 850 nm.

9. The method of claim 6, wherein the administering step is performed by intratumoral administration, subdermal administration, subcutaneous administration, parenchymal administration, or a combination thereof.

10. A method of identifying malignant cancer cells in an organism, the method comprising:
(a) administering an imaging composition to an organism, wherein the imaging composition comprises an imaging agent, and wherein the administering comprises contacting sentinel lymph nodes of the organism with the imaging agent,
wherein the imaging agent is 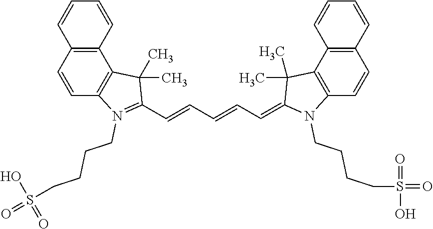
or a salt, solvate, hydrate, polymorph, or prodrug, thereof;
(b) irradiating the sentinel lymph nodes at a wavelength absorbed by the imaging agent;
(c) detecting a signal from the imaging agent to identify the sentinel lymph nodes;
(d) analyzing the identified sentinel lymph node using frozen section or conventional histopathology; and
(e) determining if the sentinel lymph nodes removed in the biopsy contain malignant cancer cells.

11. The method of claim 10, wherein the administering step is performed by intratumoral administration, subdermal administration, subcutaneous administration, parenchymal administration, or a combination thereof.

12. A method of performing angiography in an organism, the method comprising:
(a) administering an imaging composition to an organism, wherein the imaging composition comprises an imaging agent, and wherein the administering comprises contacting blood vessels, blood vessels of lymph nodes, and/or coronary lumens of the organism with the imaging agent,
wherein the imaging agent is 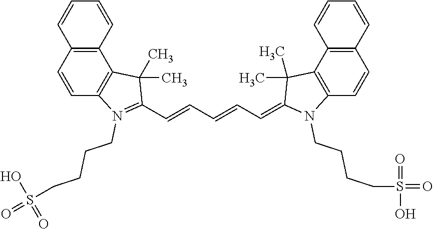
or a salt, solvate, hydrate, polymorph, or prodrug, thereof;
(b) irradiating the blood vessels, lymph nodes, and/or coronary lumens at a wavelength absorbed by the imaging agent; and
(c) detecting a signal from the imaging agent to identify the blood vessels, lymph nodes, and/or coronary lumens.

13. The method of claim 12, wherein the administering step is performed by intravenous administration.
